(19) United States
(12) Patent Application Publication
(10) Pub. No.: US 2026/0146230 A1
(43) Pub. Date: May 28, 2026

(54) CULTURE METHOD AND APPLICATION FOR PORCINE LIMBAL EPITHELIAL STEM CELLS

(71) Applicant: QINGDAO KANGMINGBEI JIAN BIOPHARMACEUTICAL CO., LTD., Qingdao City (CN)

(72) Inventors: Wenhua XU, Qingdao City (CN); Zhen SHANG, Qingdao City (CN); Xiaoying KONG, Qingdao City (CN); Dan HAN, Qingdao City (CN); Liang ZHANG, Qingdao City (CN); Nailong PAN, Qingdao City (CN); Xiaomin WANG, Qingdao City (CN); Yan TANG, Qingdao City (CN); Xiaotong LI, Qingdao City (CN)

(73) Assignee: QINGDAO KANGMINGBEI JIAN BIOPHARMACEUTICAL CO., LTD., Qingdao City (CN)

(21) Appl. No.: 19/215,422

(22) Filed: May 22, 2025

(30) Foreign Application Priority Data
Nov. 25, 2024 (CN) 2024116868234

(51) Int. Cl. C12N 5/0797

(52) U.S. Cl. CPC C12N 5/0623; C12N 2500/02; C12N 2500/60; C12N 2501/50; C12N 2501/734; C12N 2501/999

(57) ABSTRACT

The invention relates to cell biology and regenerative medicine, specifically to a culture method and application for porcine limbal epithelial stem cells. The method involves pretreatment of cells, coating of culture dishes, centrifugation, culturing, passaging, and identification by immunofluorescence staining. The cultured cells are applicable in repairing corneal diseases, including limbal stem cell deficiency. During repair, the cells can be combined with biological materials to form a stem cell engineering carrier for implantation at the injury site. The separation and culture method is simple, yields a large number of cells, and is suitable for use with porcine eyeballs. Limbal epithelial stem cells cultured in vitro form distinct cell clusters, referred to as stem cell islands. The obtained cells exhibit phenotypes including BMI1+, ΔNp63α+, CK14+, and ABCG2+. Based on experimental and clinical research, this method provides a promising approach for cell transplantation in ocular surface reconstruction.

### TECHNICAL FIELD

The invention belongs to the field of cell biology and regenerative medicine technology, and specifically relates to a culture method and application for porcine limbal epithelial stem cells to repair cells of corneal diseases including limbal stem cell deficiency (LSCD).

### BACKGROUND ART

LSCD is a complex disease that can lead to vision loss and even blindness. There are many ways to treat corneal blindness; for example, a patent was disclosed in China.

A flexible piezoelectric corneal contact lens for corneal injury repair is disclosed in Chinese patent 202211138120.9, which includes the first outer packaging film, the first inner packaging film, the piezoelectric sensing layer, the electrode pair, the second inner packaging film and the second outer packaging film from top to bottom, the flexible piezoelectric corneal contact lens also includes a rectifier, which is used to electrically connect the piezoelectric sensing layer and the electrode pair; the piezoelectric sensing layer is used to convert the energy generated during blinking into a pulse voltage signal, the rectifier is used to convert the pulse voltage signal collected by the piezoelectric sensing layer into DC and output to the electrode pair, the electrode pair is used to convert the DC signal into an external enhanced electric field to improve the endogenous electric field; the piezoelectric sensing layer is a piezoelectric electret film with a 12-angle star-shaped snowflake structure, the piezoelectric electret film is composed of three layers, namely the top electrode layer, the intermediate organic material layer and the bottom electrode layer, which constitute a flat capacitor. The pattern of the electrode pair is two concentric circles, and there is a connection line between the two concentric circles, the circumference line and the connection line of the two circles are serpentine lines. Eye drops suitable for limbal stem cell deficiency disclosed in Chinese patent 202010496058.5 includes the following components: human adipose stem cell exosomes 1-40 mg/L, excipients: sodium hyaluronate 0.5-2 g/L, vitamin B6 0.5-3 g/L, benzalkonium chloride 0.05-0.3 g/L, the remainder is medical saline, the pH value is 6.5-7.5, the preparation method includes the following steps: (1) 3-5 generations of human adipose stem cells are selected, the stem cell culture medium is used to culture cells to 70%-80% fusion, the medium is replaced with stem cell serum-free medium, the stem cells are continued to be cultured for 36 h-48 h, the culture supernatant is collected, the exosomes of human adipose stem cells are extracted, and the exosomes are resuspended to prepare the human adipose stem cell exosome solution; (2) The human adipose stem cell exosome solution prepared in step (1) is mixed with other components according to the concentration of the exosome component of the eye drops described in claim 1, and the pH is adjusted to 6.5-7.5 to obtain the eye drops, in step (1), extracting exosomes by ultra-speed gradient centrifugation is as follows: the specific centrifugal force is selected as 300×g, 20 min, and the supernatant is retained; 10000×g, 30 min, the supernatant is retained; 100000×g, 1 h, the supernatant is retained, and the precipitate is retained as human adipose stem cell exosomes. In step (2), according to the components of the eye drops described in claim 1, the sodium hyaluronate is dissolved into the medical normal saline, and then the corresponding concentration of vitamin B6 and benzalkonium chloride are dissolved in turn, the pH is adjusted to 6.5-7.5, and then the human adipose stem cell exosome solution prepared by the corresponding concentration step (1) is added, and the eye drops are prepared by mixing evenly. However, the therapeutic effect of the above schemes is not ideal. Practical studies have found that human donor cornea transplantation, which relies on healthy donor corneas, exquisite surgical techniques, and advanced medical equipment, is an effective method for the treatment of corneal blindness, however, the severe shortage of donor corneas limits the popularity of this treatment method, because a donor cornea can only meet the needs of about 70 patients. In addition, allografts have the risk of immune rejection and infection, especially in patients with inflammation and severe keratopathy.

Studies have shown that LSCD requires specialized stem cell therapy to restore eye health and vision, and cannot rely solely on traditional donor corneal grafts. The limbus contains a group of self-renewing stem cells called limbal epithelial stem/progenitor cells (LSC), which are responsible for maintaining the integrity of the corneal surface and the continuous renewal of the corneal epithelium. In the steady state, LSC maintains a slow cell cycle, and after corneal injury, the limbal homeostasis is disturbed by stimulating factors, and LSC begins to proliferate. LSC can proliferate through symmetric division and asymmetric division, in asymmetric division, LSC splits to produce a progeny stem cell to supplement the stem cell pool, as well as an undifferentiated progenitor cell with limited proliferation potential. Under normal physiological conditions, LSC migrates to the central cornea after division and proliferation, and produces terminally differentiated cells, which eventually fall off from the corneal surface due to biological functions such as blinking. LSC is supported and regulated by the corneal limbal niche, which is a highly controlled microenvironment of the human eye, when LSC is lost or the corneal limbal niche is destroyed, resulting in a decrease in the number or function of LSC, it may lead to the destruction of the corneal limbal barrier, delayed healing of corneal epithelial wounds, corneal neovascularization, inflammatory infiltration, and scar formation. Based on this, the key to the treatment of LSCD is to reconstruct the limbal niche by supplementing limbal epithelial stem cells and restoring corneal health and function.

At present, the study of LSC is limited by the lack of specific markers, although people have been looking for specific markers that can directly determine LSC, since these markers are expressed in central corneal epithelial cells and conjunctiva in recent years, one or a group of LSC absolute specific marker molecules has not been found. This limitation means that it is impossible to accurately identify corneal epithelial stem cells through existing markers, which poses a challenge to the research fields of isolation, culture, and transplantation of the limbal stem cells. At present, LSC is identified by a combination of relatively specific markers distributed in different cell locations.

### SUMMARY

The purpose of this invention is to overcome the shortcomings of the existing technology, a culture method and application for porcine limbal epithelial stem cells are developed and designed to repair the cells of corneal diseases such as LSCD.

In order to achieve the above purpose, the specific process of a porcine limbal epithelial stem cell culture method involved in the invention is as follows:

(1) Pretreated tissue

Firstly, in a biological safety cabinet, the pig eyeball tissue is immersed in a pre-cooled PBS solution (phosphate buffered salt solution) containing 10% antibiotics and pH=7.4, and rinsed three times repeatedly, each time lasting for 3 to 5 minutes;
then, the cornea is cut off, the iris attached to the cornea is removed, the limbal ring is removed, the limbal ring is placed in a solution of disperse enzyme II with a concentration of 1.2-2 U/ml, and digestion is performed in a refrigerator at a temperature of 4° C. for 14-16 hours;
finally, the epithelial cell layer of the limbal ring is isolated and transferred to an incubator at a temperature of 37° C. , and digested with trypsin for 5 minutes;
where the disperse enzyme II is a neutral protease, which can accurately act on the basement membrane region, destroy the anterior elastic layer of the cornea, realize the rapid, effective, and mild separation of the epithelium and the cornea, and maintain the integrity of the epithelial cells, its interference with temperature, pH value and serum composition shows extremely high stability, and has little effect on the epithelial cells, so as to separate the epithelial cells from the stromal layer without damaging the epithelial cells;

(2) Coated culture dish
firstly, laminin (recombinant LN521, BIOLAMINA) is diluted into 5 ug/ml laminin coating solution with 1×DPBS (Dulbecco's phosphate buffer) containing Ca++ and Mg++;
then, 2 ml of the laminin coating solution is added to the culture dish to be coated;
finally, the sealed culture dish is incubated overnight at a temperature of 2-8° C.;
where the coating concentration of LN521 coated culture dishes is 0.5-1 ug/cm²;

(3) Preliminary centrifugation
firstly, the suspension is beaten and dispersed until there is no obvious cell sheet, further digestion is prevented by adding a stem cell complete medium, and continuous centrifugation is performed at 1000 g for 5 minutes;
then, the supernatant is discarded, and 1 ml of the stem cell complete medium is added to beat and mix well, and the cell suspension is filtered to adjust the cell density to 1×10⁶/ml;
finally, the cells were seeded into a culture dish (LN521 coated) pre-added with complete culture medium and incubated in a CO2 incubator.

### (4) Cell Culture and Generation Passage

When the cell density reaches 70-80%, the stem cell complete medium, 0.25% trypsin, and PBS solution are preheated to 37° C., and the old complete medium is removed, the cells are washed twice with the PBS solution, and digested with trypsin;
after the cells are retracted and dispersed, the complete medium is added to terminate the digestion, and the cells are transferred to the EP tube (Eppendorf centrifuge tube) and centrifuged at 1000 g for 5 minutes, and the supernatant is discarded, the cells are resuspended with the complete medium, and ½ is taken and added to a new culture dish, after mixing, the cells are cultured in a CO₂ incubator, and the obtained cells are the first generation cells (P1);
the cells are subcultured again, and the obtained cells are the second generation cells (P2), similarly, the inoculation concentration of subcultured cells is 0.5×10⁴/cm²-1.0×10⁴/cm²;
where the culture conditions of CO₂ incubator are as follows: the temperature is 37° C., the CO₂ saturation is 5%, the humidity is 95%.

The antibiotic involved in the invention is an antibiotic containing 100 U/mL of penicillin and 100 μg/mL of streptomycin, referred to as double antibody;
the stem cell complete medium is MSCBM (human mesenchymal stem cell basal medium, Dayou) containing 5% serum replacement (SUPERGROW® cell culture additive, Dakewei) and 1% antibiotics.

The invention relates to a porcine limbal epithelial stem cell, which is identified by cell immunofluorescence staining, the specific process of identification is as follows:
firstly, the cell climbing pieces are placed in a six-well plate, and 1 ml of 5 ug/ml laminin coating solution is added to each well, the plate is sealed and incubated overnight at a temperature of 2-8° C.;
then, the first-generation cells and the second-generation cells are subjected to standard digestion, and the obtained cell suspension is transferred to a well plate;
the well plate is cultured in a humid environment with a temperature of 37.0° C. and a CO₂ saturation of 5% for 12-24 hours, so that the cell coverage rate reaches 60-70%.

Finally, four relative specific antibodies are selected: BMI1 (CST, 6964T) and ABCG2 (Santa Cruz Animal Health, sc-18841), ΔNp 63α (CST, 13109T), and CK14 (Wuhan Sanying, 60320-1-IG), two antibodies are paired, and a double immunofluorescence staining is performed to identify the stem cell markers.

where BMI1 is highly expressed in limbal epithelial basal cells and is a specific biomarker molecule for identifying LSC; ABCG2 is a relatively specific marker of the limbal stem cells. Keratin K14 is often present in the form of dimers with other keratins and is expressed in the basal cells of the limbal epithelium as a positive marker molecule for LSC; ΔNp63α is located in the limbus of the cornea and is not expressed in corneal epithelial cells, it is a relatively specific marker widely used at present; DAPI is used to label the nucleus.

The porcine limbal epithelial stem cells involved in the invention are applied to the repair of corneal diseases including the limbal stem cell deficiency. The repair method is simple and easy: it is compounded with biological materials into stem cell engineering carriers and implanted into the injured site, which can be regenerated and repaired.

Compared with the existing technology, the invention is suitable for separation from the eyeballs of large animals: pigs, the separation and culture methods are simple and the amount of cells obtained is large, the limbal epithelial stem cells cultured in vitro can form cell clusters, which are called the stem cell islands, the obtained limbal epithelial stem cells have the phenotypes of BMI1+, ΔNp63α+, CK14+, and ABCG2+, which can be applied to experimental research and clinical research, providing a new way for cell transplantation.

### BRIEF DESCRIPTION OF THE DRAWINGS

FIG. 1 is a schematic diagram of separating pig cornea and pig eyeball in a biological safety cabinet.

FIG. 2 is a schematic diagram of the limbal ring obtained after peeling the central cornea under a stereomicroscope.

FIG. 3 is a schematic diagram of the growth state of the P0 generation cells of the present invention after being cultured in a CO2 incubator. FIG. 3a is the growth state of the PI generation cells on the first day; FIG. 3b is the growth state of the PI generation cells on the 3rd day.

FIG. 4 is a schematic diagram of the growth state of the P1 generation cells of the present invention after being cultured in a CO2 incubator. FIG. 4a is the growth state of the PI generation cells on the 3rd day; FIG. 4 bis the growth state of the PI generation cells on the 7th day.

FIG. 5 is a schematic diagram of the results of the double immunofluorescence staining of the LSC-specific marker molecules involved in the invention. FIG. 5a is a cell immunofluorescence staining diagram of cytokeratin 14 (CK 14 ); FIG. 5b is a cell immunofluorescence staining diagram of ΔNp63α subtype in the ΔNp63 (ΔNp63α, β and γ) lacking the N-terminal domain of the cell cycle regulatory factor p63 family; FIG. 5c is a schematic diagram of the DAPI staining nuclear markers; FIG. 5d is the co-localization of CK14, ΔNp63α and DAPI.

FIG. 6 is a schematic diagram of the results of the double immunofluorescence staining of LSC-specific marker molecules involved in the invention. FIG. 6a is a cell immunofluorescence staining diagram of the polycomb gene BMI1; FIG. 6b is the cell immunofluorescence diagram of the ATP-binding cassette transporter family member ABCG2; FIG. 6c is a schematic diagram of the DAPI staining nuclear markers; FIG. 6d is the co-localization diagram of BMI1, ABCG2, and DAPI.

### DETAILED DESCRIPTION OF THE EMBODIMENTS

The following is a further explanation of the invention in combination with the attached diagrams and the specific implementation methods.

### Example 1

The specific process of the porcine limbal epithelial stem cell culture method involved in this example is as follows:
(1) The fresh and intact pig eyeball tissue obtained from the local slaughterhouse is immediately placed in D-Hank's buffer at 4° C. and transported to the sterile laboratory within 1-2 hours;
(2) The porcine eyeball tissue is placed in a biological safety cabinet, and the blood stains are washed with a pre-cooled PBS solution containing 10% double antibody and repeatedly washed three times, each time lasting for 3-5 minutes;
(3) The eyeball of pig eyeball tissue is fixed, and the complete cornea is cut off along the corneoscleral limbus using ophthalmic scissors sterilized in advance by a autoclave, as shown in FIG. 1, it is placed in a sterile PBS solution or normal saline containing 10% double antibody, and the iris attached to the cornea is removed by an ophthalmic tweezer;
where the cornea is composed of five layers, after removing the anterior elastic layer, posterior elastic layer, and endodermis, the epithelial layer and stroma layer constitute the main part of the cornea, where the stroma layer accounts for 90% of the total thickness of the cornea, while the corneal epithelium is relatively thin, accounting for only 10% of the total thickness of the cornea;

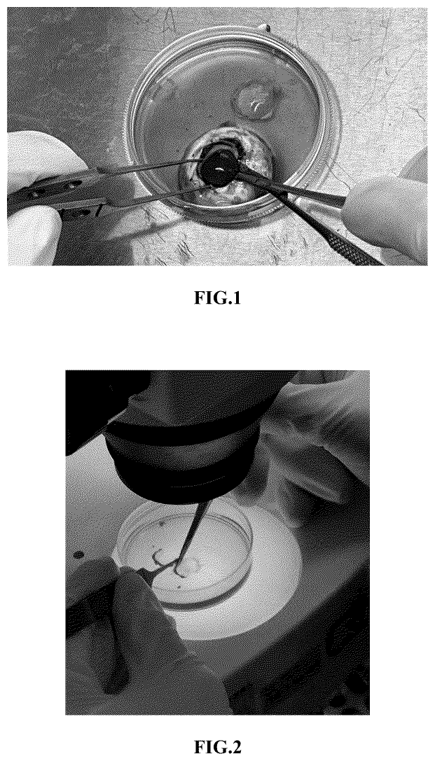

(4) Under the stereomicroscope, a small incision is cut at the edge of the corneoscleral limbus using ophthalmic scissors, and a complete central cornea is stripped along the gray-white area on the inner side of the corneoscleral limbus to obtain the limbal ring as shown in FIG. 2;

The disperse enzyme II solution is prepared with Hepe buffer salt solution (50 mM Hepes/KOH pH 7.4, 150 mM NaCl) and diluted to 1.2-2 U/ml with the stem cell complete medium;

The limbal ring is immersed in the diluted dispersion enzyme II solution and digested in a refrigerator at 4° C. for 14-16 hours;
(5) The limbal ring is placed in a culture dish containing a complete stem cell culture medium, with the assistance of a stereomicroscope, the epithelial cell layer of the limbal ring is separated by using the ophthalmic tweezer (showing a transparent film-like structure with strong refractive index), and then it is placed in an incubator at 37° C. and digested with trypsin at a concentration of 0.25% for 5 minutes.
(6) Laminin (LN521) is thawed in the refrigerator at 4° C. in advance, and then diluted with 1×DPBS containing Ca++ and Mg++ into the 5 ug/ml laminin coating solution (LN521 solution);
according to the size of the culture dish, 1-3 ml of the LN521 solution (0.5-1 ug/cm²) is added to the culture dish to be coated, the culture dish is sealed to prevent LN521 solution evaporation and external pollution, and the culture dish is incubated overnight at a temperature of 2-8° C. to ensure that the laminin evenly covers the surface of the culture dish; at the same time, the surface of the culture dish is kept moist enough to avoid laminin inactivation;
when preparing for use, the excess LN521 solution in the culture dish is absorbed, and 1 ml of the stem cell complete medium is added in advance to prevent the surface of the culture dish from drying;
(7) The cell suspension digested by trypsin is gently beaten with a pipette until the cell sheet is not visible to the naked eye;
the stem cell complete medium is added to terminate digestion, centrifuge at 1000 g for 5 minutes;
the supernatant is discarded, 1-2 ml of stem cell complete medium is added, and then it is beaten and mixed well;
the cell suspension is filtered through a 200-mesh cell filter, after the cell density is adjusted to 1×10⁶/ml, it is inoculated into a culture dish pre-coated with LN521 solution (containing 1 ml stem cell complete medium) and incubated at a temperature of 37.0° C. , a CO₂ saturation of 5% and a humidity of 95%.

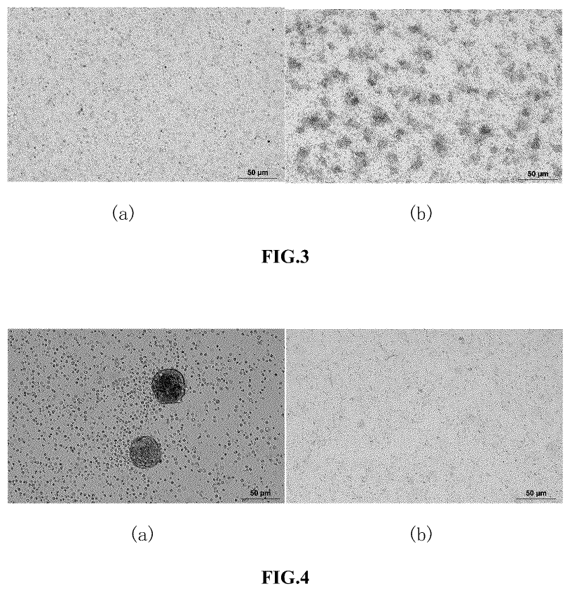

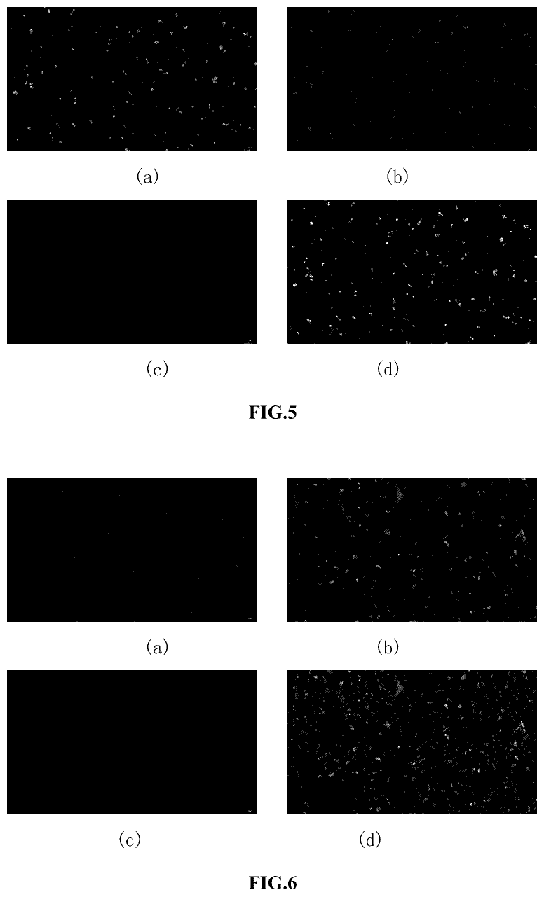

On the second day after inoculation, the adherent status of the cells is closely observed, and the fresh stem cell complete medium is replaced every 2-3 days.
(8) On the third day after culture, 85-90% fusion is achieved between the cells, as shown in FIG. 3B, the cells attached to the bottom of the culture dish are digested with 1 ml mass concentration of 0.25% trypsin for 5 min, when the cells are observed under the microscope to shrink and disperse, the same amount of the stem cell complete medium is immediately added to terminate the digestion, and the cells are transferred to the EP tube and centrifuged at 1000 g for 5 min;
the supernatant is discarded, the stem cell complete medium is added to re-suspend the cells, and ½ is added to the new culture dish, and the mixture is gently shaken;
the cells are cultured in a CO₂ incubator for 12-24 h, and the cell status is observed under an inverted microscope, if bacterial contamination is observed, the cells are discarded, and the culture is continued without pollution;
after 2-5 days, the cells gradually form a stem cell island, which is clumped and evenly distributed, after 6-10 days, the cell clumps gradually extend to the periphery, the cell state gradually relaxes, and the cell clumps gradually form cell sheets, after that, the cell sheets are fused into a complete cell layer, which is pebble-like, when the cells reach 80-90% of the fusion degree, the first generation of cells (P1) is obtained by trypsin digestion and centrifugation, and the first generation of cells (P1) is obtained after resuspension, it is transferred into another new culture dish, the third generation of cells (P3) is obtained by subculture, which still maintains a strong proliferation ability;
the cells of the P0 generation are shown in FIG. 3. FIG. 3A is the growth state on the first day, and FIG. 3B is the growth state on the third day, showing the trend of cell clusters, the cells of the P1 generation are shown in FIG. 4, FIG. 4A is the growth state on the 3rd day, forming a uniformly distributed stem cell island, FIG. 4B is the growth state on the 7th day, it has a vigorous proliferation, forms a cell layer and looks like paving pebbles.

### Example 2

The specific process of phenotypic identification of the porcine limbal epithelial stem cells involved in this example is as follows:
(1) the cell climbing pieces are placed in a six-well plate, and 1 ml of LN521 solution with a concentration of 5 ug/ml is added to each well, sealed the plate, and incubated overnight at a temperature of 2-8° C.;
(2) P1 and P2 cells are seeded onto the cell slides coated with LN521 solution;
(3) when the cell density reaches 60-70%, the excess stem cell complete medium in the plate is absorbed, 1-2 ml of 4% paraformaldehyde is added, the plate is sealed and kept at room temperature for 20 minutes, it is washed 3 times with PBS solution for 3 minutes each time;
(4) at room temperature, 0.2% Triton X-100 is used for penetrating for 5 minutes, it is washed 3 times with PBS solution for 3 minutes each time;
(5) after blocking for 30 minutes, specific primary antibodies (BMI1, ΔNp63α, CK14, ABCG2) are added and incubated overnight at 4° C.
(6) the fluorescently labeled secondary antibodies are added and incubated for 1 hour in the dark;
(7) nuclear staining is performed using DAPI, and the cells are allowed to stand at room temperature for 10 minutes;
(8) 20 μL of the anti-fluorescence quenching agent is added to the seal, it is observed under a fluorescence microscope, and the images are collected.

What is claimed is:

1. A method for culturing porcine limbal epithelial stem cells, including the following process:
(1) Pretreated tissue
firstly, in a biological safety cabinet, the pig eyeball tissue is immersed in a pre-cooled PBS solution containing 10% antibiotics and pH=7.4, and rinsed;
then, the cornea is cut off, the iris attached to the cornea is removed, the limbal ring is removed, the limbal ring is placed in a solution of disperse enzyme II with a concentration of 1.2-2 U/ml, and digestion is performed in a refrigerator at a temperature of 4 °C for 14-16 hours;
finally, the epithelial cell layer of the limbal ring is isolated and transferred to an incubator at a temperature of 37° C., and digested with trypsin for 5 minutes;
(2) coated culture dish
firstly, laminin is diluted into 5 ug/ml laminin coating solution with 1×DPBS (Dulbecco's phosphate buffer) containing Ca++ and Mg++ ;
then, 2 ml of the laminin coating solution is added to the culture dish to be coated;
finally, the sealed culture dish is incubated overnight at a temperature of 2-8° C.;
(3) preliminary centrifugation
firstly, the suspension is beaten and dispersed until there is no obvious cell sheet, further digestion is terminated, and centrifugation is performed at 1000 g for 5 minutes;
then, the supernatant is discarded, and 1 ml of the stem cell complete medium is added to beat and mix well, and the cell suspension is filtered to adjust the cell density to 1×10⁶/ml;
finally, the cells are inoculated into a culture dish (LN521 coated) that had been pre-added with complete culture medium in humid air with a temperature of 37.0° C. and a CO₂ saturation of 5%.
(4) cell culture and generation passage
when the cell density reaches 70-80%, the stem cell complete medium, 0.25% trypsin, and PBS solution are preheated to 37° C., and the old stem cell complete medium is removed, the cells are washed with the PBS solution and digested with trypsin;
after the cells are retracted and dispersed, the stem cell complete medium is added, the digestion is terminated, and the cells are transferred to the EP tube and centrifuged at 1000 g for 5 minutes, and the supernatant is discarded, the cells are resuspended, and ½is taken and added to a new culture dish, after mixing, the cells are cultured in a CO₂ incubator, and the obtained cells are the first generation cells (P1);
the cells are subcultured again, and the obtained cells are the second generation cells (P2), similarly, the inoculation concentration of subcultured cells is 0.5×10⁴/cm²-1.0×10⁴/cm².

2. The method for culturing porcine limbal epithelial stem cells according to claim 1, wherein the coating concentration of LN521 in the culture dish involved in step (2) is 0.5-1 ug/cm²; the culture conditions of the CO₂ incubator involved in step (4) are: the temperature is 37° C., the CO₂ saturation is 5%, and the humidity is 95%.

3. The method for culturing porcine limbal epithelial stem cells according to claim 1, wherein the antibiotic is an antibiotic containing penicillin 100 U/mL and streptomycin 100 μg/mL, referred to as double antibody; the complete medium of stem cells is MSCBM containing 5% serum replacement and 1% antibiotics.

4. The method for culturing porcine limbal epithelial stem cells according to claim 1, wherein the specific process is as follows:
(1) the fresh and intact pig eyeball tissue obtained from the slaughterhouse is immediately placed in D-Hank's buffer at 4° C. and transported to the sterile laboratory within 1-2 hours;
(2) the porcine eyeball tissue is placed in a biological safety cabinet, and the blood stains are washed with a pre-cooled PBS solution containing 10% double antibody and repeatedly washed three times, each time lasting for 3-5 minutes;
(3) the eyeball of pig eyeball tissue is fixed, and the complete cornea is cut off along the corneoscleral limbus using ophthalmic scissors sterilized in advance by a autoclave, it is placed in a sterile PBS solution or normal saline containing 10% double antibody, and the iris attached to the cornea is removed by an ophthalmic tweezer;
(4) under the stereomicroscope, a small incision is cut at the edge of the corneoscleral limbus using ophthalmic scissors, and a complete central cornea is stripped along the gray-white area on the inner side of the corneoscleral limbus to obtain the limbal ring;
the disperse enzyme II solution is prepared with Hepe buffer salt solution and diluted to 1.2-2 U/ml with the stem cell complete medium;
the limbal ring is immersed in the diluted dispersion enzyme II solution and digested in a refrigerator at 4° C. for 14-16 hours;
(5) the limbal ring is placed in a culture dish containing a complete stem cell culture medium, with the assistance of a stereomicroscope, the epithelial cell layer of the limbal ring is separated by using the ophthalmic tweezer, and then it is placed in an incubator at 37 ° C. and digested with trypsin at a concentration of 0.25% for 5 minutes;
(6) laminin is thawed in the refrigerator at 4° C. in advance, and then diluted with 1×DPBS containing Ca++ and Mg++ into the 5 ug/ml laminin coating solution;
according to the size of the culture dish, 1-3 ml of the LN521 solution is added to the culture dish to be coated, the culture dish is sealed and incubated overnight at a temperature of 2-8° C.;
when preparing for use, the excess LN521 solution in the culture dish is absorbed, and 1 ml of the stem cell complete medium is added in advance;
(7) the cell suspension digested by trypsin is gently beaten with a pipette until the cell sheet is not visible to the naked eye;
the stem cell complete medium is added to terminate digestion, centrifuge at 1000 g for 5 minutes;
the supernatant is discarded, 1-2 ml of stem cell complete medium is added, and then it is beaten and mixed well;
the cell suspension is filtered through a 200-mesh cell filter, after the cell density is adjusted to 1×106/ml, it is inoculated into a culture dish pre-coated with LN521 solution, and incubated at a temperature of 37.0° C., a CO2 saturation of 5% and a humidity of 95%;
the stem cell complete medium is replaced every 2-3 days;
(8) on the third day after culture, 85-90% fusion is achieved between the cells, the cells attached to the bottom of the culture dish are digested with 1 ml mass concentration of 0.25% trypsin for 5 min, when the cells are observed under the microscope to shrink and disperse, the same amount of the stem cell complete medium is immediately added to terminate the digestion, and the cells are transferred to the EP tube and centrifuged at 1000 g for 5 min;
the supernatant is discarded, the stem cell complete medium is added to re-suspend the cells, and ½ is added to the new culture dish, and the mixture is gently shaken;
the cells are cultured in a CO₂ incubator for 12-24 h, and the cell status is observed under an inverted microscope, if bacterial contamination is observed, the cells are discarded, and the culture is continued without pollution;
the culture is continued, after 2-5 days, the cells gradually form a stem cell island, which is clumped and evenly distributed, after 6-10 days, the cell clumps gradually extend to the periphery, the cell state gradually relaxes, and the cell clumps gradually form cell sheets, after that, the cell sheets are fused into a complete cell layer, which is pebble-like, when the cells reach 80-90% of the fusion degree, the first generation of cells is obtained by trypsin digestion, centrifugation, and resuspension, it is transferred into another new culture dish, the second generation of cells is obtained by subculture.

5. The method for culturing porcine limbal epithelial stem cells according to claim 4, wherein the porcine limbal epithelial stem cells are applied to the repair of corneal diseases including limbal stem cell deficiency.

6. The method for culturing porcine limbal epithelial stem cells according to claim 5, wherein the porcine limbal epithelial stem cells and biomaterials are compounded into stem cell engineering carriers and implanted into the injured site during repair.

7. The method for culturing porcine limbal epithelial stem cells according to claim 4, wherein the porcine limbal epithelial stem cells are identified by cell immunofluorescence color, the process of identification is:
firstly, the cell climbing pieces are placed in six-well plates, and 1 ml 5 ug/ml laminin coating solution is added to each well, the plate is sealed and incubated overnight at a temperature of 2-8° C.;
then, the first-generation cells and the second-generation cells are subjected to standard digestion, and the obtained cell suspension is transferred to a well plate;
the well plate is cultured in a humid environment with a temperature of 37.0° C. and a CO₂ saturation of 5% for 12-24 hours, so that the cell coverage rate reaches 60-70%;
finally, four relative specific antibodies: BMI1 and ABCG2, ΔNp63α and CK14 are paired for double immunofluorescence staining to identify the stem cell markers.

8. The method for culturing porcine limbal epithelial stem cells according to claim 5, wherein the specific process of phenotypic identification is as follows:
(1) the cell climbing pieces are placed in a six-well plate, and 1 ml of LN521 solution with a concentration of 5 ug/ml is added to each well, the plate is sealed and incubated overnight at a temperature of 2-8° C.;
(2) P1 and P2 cells are seeded onto the cell slides coated with LN521 solution;
(3) when the cell density reaches 60-70%, the excess stem cell complete medium in the plate is absorbed, 1-2 ml of 4% paraformaldehyde is added, the plate is sealed and kept at room temperature for 20 minutes, it is washed with PBS solution;
(4) at room temperature, 0.2% Triton X-100 is used for penetrating for 5 minutes, it is washed with PBS solution;
(5) after blocking for 30 minutes, specific primary antibodies are added and incubated overnight at 4° C..
(6) the fluorescently labeled secondary antibodies are added and incubated for 1 hour in the dark;
(7) the nuclear staining is performed using DAPI, and the cells are allowed to stand at room temperature for 10 minutes;
(8) 20 μL of the anti-fluorescence quenching agent is added to the seal, it is observed under a fluorescence microscope, and the images are collected.
